(19) United States
(12) Patent Application Publication
(10) Pub. No.: US 2009/0076549 A1
(43) Pub. Date: Mar. 19, 2009

(54) ORTHOPEDIC IMPLANT SYSTEM

(75) Inventors: Roy K Lim, Germantown, TN (US); Jaredan Braal, Memphis, TN (US); Noriaki Kawakami, Nagoya (JP)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/856,314

(22) Filed: Sep. 17, 2007

(51) Int. Cl. A61B 17/70; A61B 17/56; A61B 17/04; A61B 17/58

(52) U.S. Cl. 606/246; 606/103; 606/264; 606/278; 606/301

(57) ABSTRACT

A system is provided for use in orthopedic surgery, for example to connect a spinal rod with a vertebral screw or hook. The system generally includes a receiver member for receiving an elongated member, a bone implant member, such as a bone screw, and a locking washer to assist in locking the receiver member at a desired angular position relative to the bone implant member. The receiver member and the washer may be positioned on opposite sides of the bone implant member. Additionally, the receiver member may define an aperture to receive a closure member to lock an elongated member in a passage in the receiver member.

The present disclosure relates to devices and implants used in orthopedic surgical procedures. More particularly, the present disclosure concerns orthopedic implant systems for connecting an elongated member with a bone implant member. The systems can be useful for correction of spinal injuries or deformities.

In the realm of orthopedic surgery, it is well known to use implants to fix the position of bones. In this way, the healing of a broken bone can be promoted, and malformations or other injuries can be corrected. For example, in the field of spinal surgery, it is well known to place such implants into vertebrae for a number of reasons, including (a) correcting an abnormal curvature of the spine, including a scoliotic curvature, (b) to maintain appropriate spacing and provide support to broken or otherwise injured vertebrae, and (c) perform other therapies on the spinal column.

Typical implant and connection systems include several pieces, which commonly are useful and may be associated with only specific other pieces. Bone screws, hooks, and clamps are well known as fixation devices, which are connected or adjoined to a particular bone as a connection between the bone and the connection system which can include a support and/or stabilizing member such as a spinal rod. In such a system, a series of two or more bone screws may be inserted into two or more vertebrae to be instrumented. A spinal rod is then placed within or coupled to the screws, or is placed within a connecting device that links the rod and a screw, and the connections are tightened. In certain situations, a closure device is tightened against the rod to clamp the rod in place against the bottom of a channel. The closure device may positively secure the rod in place to prevent rotational or translational movement of the rod relative to the bone screw and the bone in which it is anchored. In this way, a rigid supporting structure is fixed to the vertebrae, with the rod providing the support that promotes correction or healing of the vertebral malformation or injury by keeping the vertebrae in a particular position.

A multitude of spinal fixation systems exist; however, the systems can be difficult to assemble, position and secure. Therefore, a need exists for improved spinal fixation systems.

### BRIEF DESCRIPTION OF THE DRAWINGS

FIG. 1 is a perspective view of one embodiment of a system according to the present disclosure.

FIG. 2 is an exploded perspective view of a system according to the embodiment shown in FIG. 1.

FIG. 3 is a front view of a system according to the embodiment shown in FIG. 1.

FIG. 4 is a front cross-sectional view of a system according to the embodiment shown in FIG. 1.

FIG. 5 is a perspective view of one embodiment of a system according to the present disclosure.

FIG. 6 is an exploded perspective view of a system according to the embodiment shown in FIG. 5.

FIG. 7 is a front view of a system according to the embodiment shown in FIG. 5.

FIG. 8 is a front cross-sectional view of a system according to the embodiment shown in FIG. 5.

### DESCRIPTION OF THE PREFERRED EMBODIMENT

For the purposes of promoting an understanding of the principles of the disclosure, reference will now be made to the embodiment illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

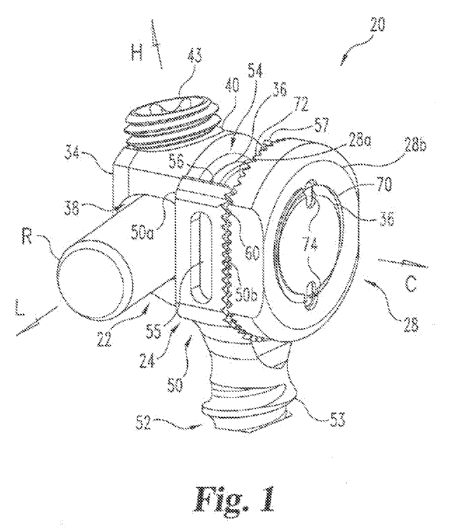

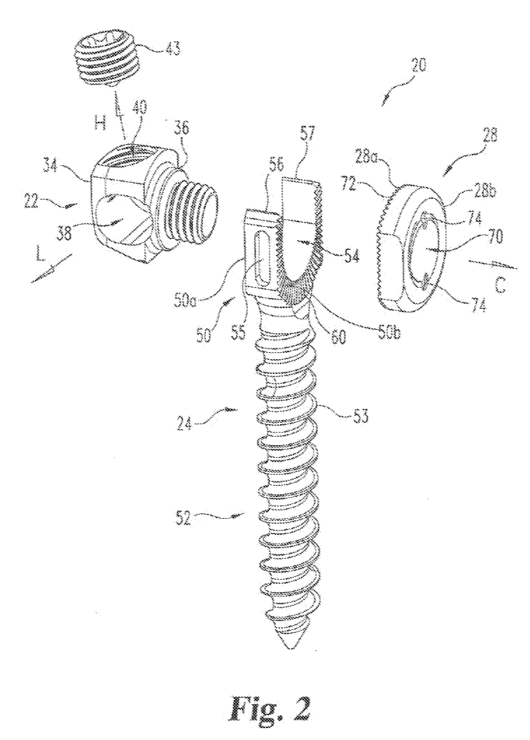

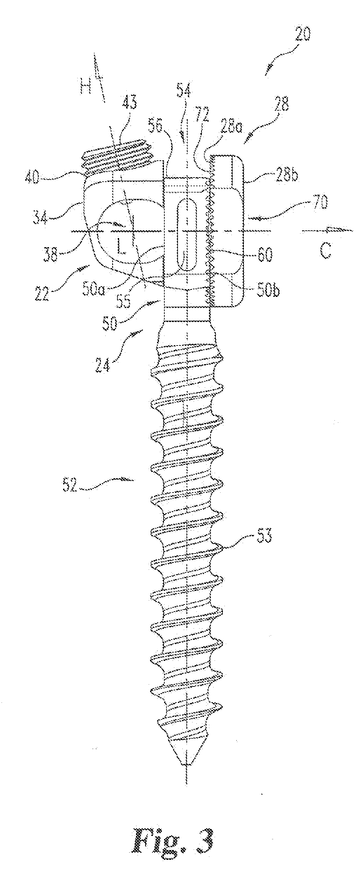

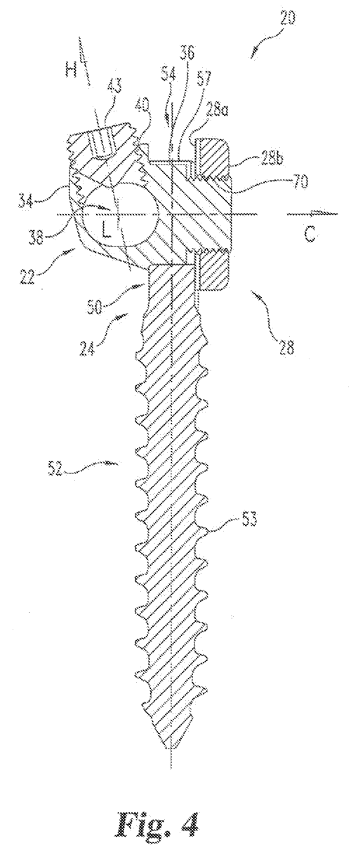

Referring generally to FIGS. 1-4, embodiments of an orthopedic implant system 20 are shown. System 20 generally includes a receiver member 22, an open-top bone implant member 24, and a disk or washer 28. As illustrated, bone implant member 24 may be positioned generally between receiver member 22 and washer 28. As will be described further below, receiver member 22 is designed to accommodate at least a part of an elongated member, such as spinal rod R. In the illustrated embodiment, bone implant member 24 is bone screw. However, it is contemplated that the bone implant member utilized within system 20 may be another appropriate bone fixation element, such as a hook, bolt or similar fixation device.

Receiver member 22, in one embodiment, includes a body portion 34 and an extension portion 36. An elongated passage 38 extends through receiver member 22 along a longitudinal axis L, and is sized to accommodate a section of an elongated member, such as spinal rod R. It should be appreciated that the elongated member utilized in conjunction with system 20 can be any appropriate elongated member such as a rod, bar, connector, or other orthopedic construct. Further, the elongated member may have one of a number of desired lengths. Aperture or hole 40 is provided in body portion 34 for a locking member. In the illustrated embodiment, hole 40 extends along a closure axis or height axis H. In certain embodiments, hole 40 is threaded and is adapted to accommodate a threaded set screw 43, to be threaded in and locked against an elongated member (e.g. rod R) within passage 38. As illustrated, set screw 43 may include an internal driving print to receive the head of a driving tool. Additionally, it should be appreciated that other appropriate closure members may be used. Further, it will be appreciated that more than one hole or aperture may be provided in another embodiment.

In certain embodiments, a hole may be provided in extension portion 36, with portion 36 being substantially hollow cylindrical in shape. In certain other embodiments, extension portion 36 is substantially solid, with no hole extending therethrough. Additionally, extension portion 36 may have a substantially uniform diameter, or may include a section of reduced diameter as in the illustrated embodiment. Extension portion 36 may include threads and be configured to engage with a threaded hole defined in washer 28 to connect receiver member 22 and washer 28 along a connection axis C. As illustrated, hole 40 may be angled such that height axis H is oblique to connection axis C. In certain other embodiments, height axis H may be perpendicular to connection axis C.

Bone implant member 24 may include a head portion 50 and a bone engaging portion 52. Bone engaging portion 52 may include coarse threads 53 readily adapted for solid fixation within the cancellous bone of a vertebral body and can terminate in a tapered tip to assist in the gradual engagement and advancement of the threads into the vertebral body. Head portion 50 includes a first surface 50a and an opposite second surface 50b, and defines a passage 54 therethrough for accommodating at least a portion of extension portion 36. In the illustrated embodiment, head portion 50 includes two branches 56 and 57 defining passage 54, such that passage 54 is a substantially U-shaped channel. Head portion 50 may optionally include one or more indentations or pockets 55 for engagement with an insertion tool.

As illustrated, receiver member 22 is positionable adjacent first surface 50a and washer 28 is positionable adjacent second surface 50b. In certain embodiments, first surface 50a may be flat so as to enable a flush connection between a part of it and a portion of a face on body portion 34 of receiver member 22, and second surface 50b may include a roughened exterior or other such surface feature amenable to making a secure connection with washer 28. As illustrated, second surface 50b may include splines 60 configured to matingly engage splines disposed on washer 28. In such embodiments, splines 60 may extend substantially from passage 54 to an outer edge of second surface 50b.

Washer 28 includes a first surface 28a and a second surface 28b, and defines a hole 70 therethrough to receive at least a portion of extension portion 36 of receiver member 22. In the illustrated embodiment, hole 70 is threaded and thereby configured to threadingly engage extension portion 36. In such embodiments, upon threaded advancement of extension portion 36 in hole 70, receiver member 22 is substantially not rotatable with respect to washer 28. In certain other embodiments, hole 70 and extension portion 36 may be keyed together or both may have a similarly dimensioned square, rectangular, polygonal, rounded, tapered or other shape to ease the fitting together of receiver member 22 and washer 28, and/or to limit or prevent rotation with respect to each other. In the illustrated embodiment, hole 70 extends from first surface 28a to second surface 28b. In certain other embodiments, hole 70 extends only partially through a width of washer 28 from first surface 28a.

Washer 28 may be generally ring shaped with substantially flat end sections, although it may be of any of a variety of shapes or configurations. In certain embodiments, first surface 28a of washer 28 includes a roughening surface feature, such as radial splines 72 configured to engage splines 60 on second surface 50b of head portion 50. In such embodiments, splines 72 may extend substantially from hole 70 an outer edge of first surface 28a. Washer 28 may further include recesses 74 machined into washer 28 to assist in enhancing the connection between extension portion 36 and washer 28, as will be described further below.

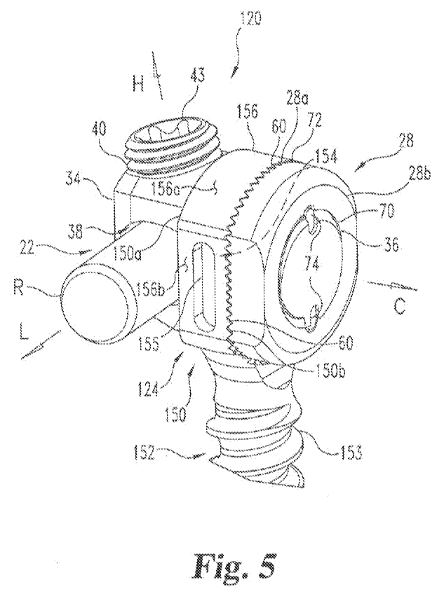

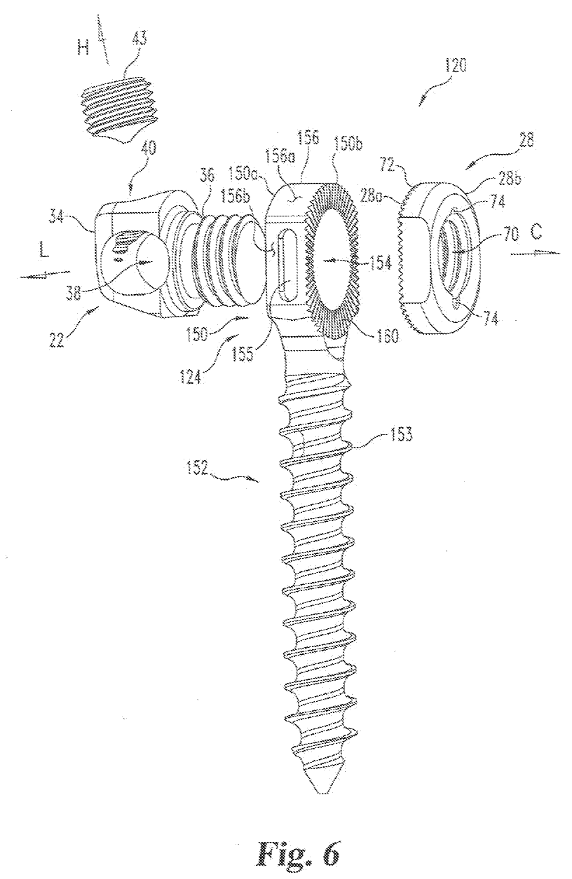

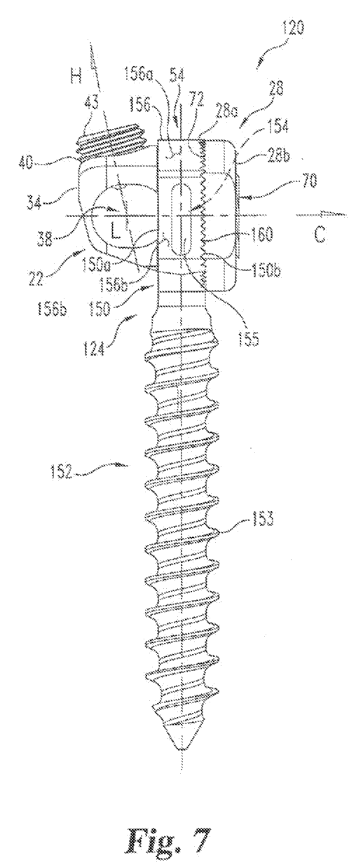

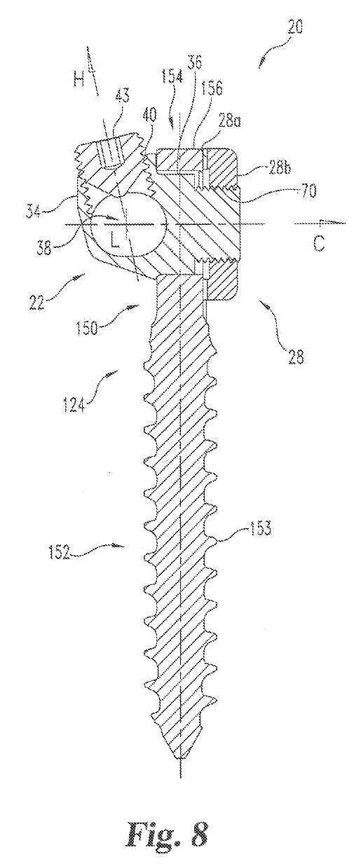

Referring generally to FIGS. 5-8, embodiments of an orthopedic implant system 120 are shown. System 120 is similar to system 20 in configuration, assembly, and use, with system 120 including a closed-top bone implant member 124, alternatively to an open-top bone implant member. System 120 also generally includes receiver member 22 and washer 28, as described above in connection with system 20, positionable on opposite sides of bone implant member 124.

Bone implant member 124 includes a head portion 150 and a bone engaging portion 152. Bone engaging portion 152 may include coarse threads 153 readily adapted for solid fixation within the cancellous bone of a vertebral body and can terminate in a tapered tip to assist in the gradual engagement and advancement of the threads into the vertebral body. Head portion 150 includes a body 156 having a first surface 150a and an opposite second surface 150b. Body 156 of head portion 150 defines a passage 154 therethrough for accommodating at least a portion of extension portion 36 of receiver member 22. Similar to system 20, extension portion 36 is configured to be received in passage 154 and threadingly engage washer 28 adjacent second side 150b of head portion 150. In the illustrated embodiment, body 156 is configured such that passage 154 is a closed channel. In certain embodiments, body 156 is substantially ring-shaped, with curved sections 156a and substantially flat end sections 156b. Head portion 150 may optionally include one or more indentations or pockets 155 for engagement with an insertion tool.

As illustrated, receiver member 22 is positionable adjacent first surface 150a and washer 28 is positionable adjacent second surface 150b. In certain embodiments, first surface 150a may be flat so as to enable a flush connection between a part of it and a portion of a face on body portion 34 of receiver member 22, and second surface 150b includes splines 160 configured to matingly engage splines 72 disposed on first surface 28a of washer 28. In such embodiments, splines 160 may extend substantially from passage 154 to an outer edge of second surface 150b.

Generally referring to FIGS. 1-4 regarding system 20 and FIGS. 5-8 regarding system 120, the assembly, operation and use of the systems will be described with reference to a surgical procedure involving a section of spine of a patient. It should be appreciated that other uses of system 20 and/or 120 described herein and other surgical procedures can be made.

In certain embodiments, partial assembly of the systems described herein may occur prior to implantation of the particular system into the patient so that the system may be quickly and easily implanted. Regarding system 20, receiver member 22 may be threadingly engaged with washer 28 prior to implantation of the system. Once assembled, receiver member 22 and washer 28 may have their connection enhanced to impede or prevent disassembly, as further discussed below. Thereafter, receiver member 22 and washer 28 may be engaged with bone implant member 24 by positioning part of extension portion 36 into passage 54. It is contemplated that in other embodiments, at least partial assembly of the systems described herein can occur during the surgical procedure and/or substantially simultaneously with implantation of part of the particular system.

To treat the condition or injury of the patient, the surgeon obtains access to the surgical site in any appropriate manner, e.g. through incision and retraction of tissues. It is contemplated that the systems discussed herein can be used in minimally-invasive surgical techniques where the spinal segment is accessed through a micro-incision, a sleeve, or one or more retractors that provide a protected passageway to the area. The systems discussed herein also have application in open surgical techniques where skin and tissue are incised and retracted to expose the surgical site.

Once access to the surgical site has been obtained, e.g. via an opening such as a midline incision above the affected area, with tissue being resected, or by other surgical procedure, the surgeon may implant one or more bone implant members adjacent vertebrae of a spinal segment that require compression, distraction and/or support in order to relieve or improve their condition. In certain embodiments, pilot holes in vertebrae may be made and threaded bone-engaging portions of the bone implant members may be inserted into or otherwise connected to a vertebral body. The bone engaging portions may be threaded or otherwise inserted into the vertebrae to a desired depth and/or desired orientation relative to a longitudinal axis of the spinal segment. Bone engaging portions 52 and 152 of bone implant members 24 and 124, respectively, may be fixed in or on a vertebra in such appropriate manners. The surgeon or other medical professional can use a driving tool or other similar instrument to drive the bone engaging portion into the vertebra. In certain embodiments, the insertion tool may include portions configured to be received in and engage indentions 55 and/or 155, if present, to assist in the insertion of bone engaging portions 52 and/or 152 into bone.

In certain embodiments, receiver member 22 may first be engaged with bone implant member 24 and/or 124 prior to threaded engagement of washer 28 and receiver member 22. In such embodiments, extension portion 36 is received in passage 54 and/or 154, such that part of extension portion 36 extends beyond passage 54 and/or 154 for threaded engagement with washer 28. Receiver member 22 is positioned adjacent first surface 50a and/or 150a of head portion 50 and/or 150. Extension portion 36 may be threadingly advanced into threaded hole 70 in washer 28 along connection axis C, such that first surface 28a of washer 28 is positioned adjacent second surface 50b and/or 150b of head portion 50 and/or 150. As illustrated, splines 60 and/or 160 of head portion 50 and/or 150 and splines 72 of washer 28 face each other upon assembly of the systems. It is contemplated that receiver member 22 and washer 28 may be engaged with bone implant member 24 and/or 124 either before or after the bone implant member is inserted into bone.

At this point, receiver member 22 may be rotatable with respect to bone implant member 24 and/or 124 around connection axis C. In such embodiments, at this point, splines 60 and/or 160 of bone implant member 24 and/or 124 are not engaged with splines 72 of washer 28. Accordingly, first surface 28a of washer 28 and second surface 50b and/or 150b of head portion 50 and/or 150 are separable by a sufficient space to allow washer 28 to also rotate with respect to bone implant member 24 and/or 124. In certain embodiments, this may be thought of as a first, unlocked or open configuration of system 20 and/or 120. Accordingly, receiver member 22 may be positioned at a desired angular position relative to bone implant member 24 and/or 124.

In certain embodiments, upon assembly of system 20 and/or 120, the connection between extension portion 36 and washer 28 may be enhanced by staking threads of extension portion 36 to threads adjacent hole 70. In such embodiments, the threads of extension portion 36 are deformed by a pressing operation, urging the material of the threads of extension portion 36 into recesses 74 in washer 28, substantially preventing washer 28 from unthreading or otherwise disengaging from extension portion 36. In certain other embodiments, the connection between extension portion 36 and washer 28 may be enhanced in other appropriate ways such as by drilling, pinning, or welding, as examples.

An elongated member, such as spinal rod R, may be placed in a longitudinal channel in the receiver member, such as passage 38 of receiver member 22 along longitudinal axis L. Before or after spinal rod R is positioned in passage 38, receiver member 22 may be rotated relative to bone implant member 24 and/or 124 to achieve the desired angular position between the components of system 20 and/or 120. In certain embodiments, the spinal rod may be received in a channel or passage of another receiver member engaged with another bone implant member inserted into another vertebra to secure a section of vertebrae of a spinal segment. In many instances of spinal surgery, a surgeon will orient and/or bend the elongated members, or spinal rods, so that the members are positioned substantially parallel to a portion of the spine. Any final adjustments the surgeon desires, such as adjustment to the positions of vertebral bodies, part(s) of system 20 and/or 120, or the elongated member or bone fixation member, may be made. In certain embodiments, once any and all adjustments are made to reach the proper and desired positioning, the elongated member can be secured with reference to the bone implant member.

A locking member such as set screw 43 may be inserted into hole 40 in receiver member 22 along height axis H. In certain embodiments, hole 40 is threaded and set screw 43 is threadingly received into hole 40. It will be understood that other types of appropriate locking members may be used to secure and lock system 20 and/or 120. Set screw 43 or other appropriate locking member is tightened against onto the elongated member and presses the elongated member, such as spinal rod R, down toward the bottom of passage 38 and/or against first surface 50a and/or 150a of head portion 50 and/or 150 of system 20 and/or 120. In certain embodiments, as illustrated, the oblique angle between height axis H and connection axis C may better enable the elongated member to sufficiently press against first surface 50a and/or 150a of head portion 50 and/or 150. The elongated member contacts first surface 50a and/or 150a of head portion 50 and/or 150 of bone implant member 24 and/or 124, thereby forcing the bone implant member toward and against washer 28. Accordingly, splines 60 and/or 160 disposed on second surface 50b and/or 150b non-rotatably engage splines 72 disposed on first surface 28a of washer 28, thereby substantially preventing rotational movement of washer 28 and receiver member 22 relative to bone implant member 24 and/or 124 and locking the receiver member at the desired angular position relative to the bone implant member. Accordingly, system 20 and/or 120 is locked, and the elongated member and bone implant member or other bone attachment member are immobilized with respect to each other. In certain embodiments, this may be thought of as a second, locked configuration of system 20 and/or 120.

In certain embodiments, system 20 and/or 120 may be oriented in the patient such that the spinal rod is medial to the bone implant member. In other words, the spinal rod may be nearer to the spinous processes than the bone implant member. Additionally, in certain situations, such as those involving pediatric patients for example, system 20 and/or 120 may possibly reduce the necessary removal of adjacent bone or tissue and/or reduce the likelihood of trauma and/or damage thereto.

While one example of the use of system 20 and/or 120 described above is in the spinal field, it will be appreciated that the disclosed systems can be used in a variety of orthopedic surgical procedures and/or with a variety of orthopedic surgical implants. Of course, any of system 20 and/or 120 can be sized or otherwise configured to be used in one or more parts of the spine (e.g. cervical, thoracic, lumbar or sacral), or with spinal implants such as intervertebral cages or spacers, rod connectors, bone plates, bone-growth promoting substances, or with other devices or therapeutic substances. The parts described herein may be made of biocompatible materials such as stainless steel, titanium, certain hard plastics, shape-memory materials and/or other sturdy materials. Further, while individual embodiments of a system have been described herein, it is understood that features, parts or aspects of one embodiment could be incorporated into another embodiment.

Additionally, while the implantation of system 20 and/or 120 has been described above as inserting an elongated member (e.g. a rod) into the receiver member after implantation of the particular system, it will be appreciated that other methods can be used. For example, a surgeon may pre-fit one or more components of system 20 and/or 120 to a rod or other spinal implant device before inserting such systems into the body or placing such systems on or with other implant devices. Thus, after bone implant members are placed on or in the spine or at another surgical site, a rod with such pre-fitted components can be placed in the surgical site so that the pre-fitted components connect to respective bone implant members. Similarly, such pre-fitted components could have bone implant members pre-connected to them. Once the surgical site is properly prepared, the rod with such pre-fitted components can be introduced, with bone implant members connected to vertebrae or other bones, appropriate adjustments made to bone, receiver members, rod and/or bone implant members, and the systems locked when such adjustments are completed.

Any or all of the systems described herein can be provided in a sterile pack for providing necessary parts, or a variety of parts, to a surgeon. For example, one or more predetermined types or sizes of receiver member, bone implant member, washer and/or set screw may be provided in a single sterile package or kit. A surgeon can choose the sizes or types of systems and/or components he or she wishes to use during surgery. Alternatively, sterile kits containing a single predetermined size or type of receiver member, bone implant member, washer and/or set screw may be provided, so that waste of unneeded parts is limited or eliminated. Packages or kits of the structures described herein can also include or be provided with spinal rods, instrumentation (e.g. rod benders, cutters, pushers, reducers or other rod handling tools; screwdrivers or other implanting tools; forceps, grippers or other holding tools; drills, bits, awls, reamers, guides, taps or other hole making tools; gauges or other measuring or sizing tools; or others) or other implants or devices useful in orthopedic surgery.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A spinal fixation system, comprising:
a receiver member having a body portion and an extension portion, said extension portion extending along a connection axis, wherein said body portion defines a passage extending along a longitudinal axis to receive part of an elongated member, said connection axis being transverse to said longitudinal axis, wherein said body portion further defines at least one aperture in communication with said passage to receive a closure member to lock an elongated member in said passage;
a bone implant member having a bone engaging portion and a head portion having a first surface and a second surface, wherein said head portion defines a passage configured to receive part of said extension portion, said first surface of said head portion being positioned adjacent said body portion of said receiver member for contacting an elongated member in said passage of said receiver member, and said second surface of said head portion facing away from said body portion of said receiver member; and
a washer having a first surface and a second surface, said first surface of said washer positioned adjacent said second surface of said head portion of said bone implant member wo that said head portion is between said washer and said body portion of said receiver member;
wherein said extension portion of said receiver member is received in said passage of said head portion of said bone implant member and engages said washer, said receiver member being rotatable relative to said bone implant member along said connection axis prior to locking the system.

2. The system of claim 1, wherein said second surface of said head portion is roughened, wherein said first surface of said washer is roughened and configured to engage said second surface of said head portion.

3. The system of claim 2, wherein said second surface of said head portion and said first surface of said washer each include radial splines.

4. The system of claim 1, wherein said washer includes a hole extending along said connection axis from said first surface toward said second surface, said hole including threads and being configured to receive part of said extension portion, wherein said extension portion of said receiver member includes external threads configuring to engage said threads of said hole along said connection axis.

5. The system of claim 1, wherein said aperture in said receiver member includes threads to engage a closure member.

6. The system of claim 1, comprising a closure member configured to be received in said aperture.

7. The system of claim 6, wherein said closure member is configured to press an elongated member against said first surface of said head portion, thereby pressing said head portion against said washer, to substantially lock the elongated member in said passage in said receiver member and substantially lock said receiver member relative to said bone implant member at a desired angular position.

8. The system of claim 6, wherein said closure member is a set screw.

9. The system of claim 1, wherein said head portion of said bone implant member includes two opposing branches defining said passage in said head portion, said passage in said head portion being a substantially U-shaped channel.

10. The system of claim 1, wherein said head portion of said bone implant member is substantially ring shaped with said passage extending along said connection axis.

11. The system of claim 1, wherein said aperture extends along a closure axis, wherein said aperture is angled such that said closure axis is oblique to said connection axis.

12. The system of claim 1, wherein said bone implant member is a bone screw and said bone engaging portion includes threads.

13. A spinal fixation system, comprising:
a receiver member having a body portion and an extension portion, said extension portion extending along a connection axis, wherein said body portion defines a passage extending along a longitudinal axis to receive part of an elongated member, said connection axis being transverse to said longitudinal axis, wherein said body portion further defines at least one aperture in communication with said passage to receive a closure member to lock an elongated member in said passage;
a bone implant member having a bone engaging portion and a head portion having a first surface and a second surface, wherein said head portion defines a passage configured to receive part of said extension portion; and
a washer having a first surface and a second surface, said washer being configured to engage said extension portion of said receiver member; and
a closure member configured to be received in said aperture in said body portion of said receiver member;
wherein said receiver member, said bone implant member and said washer are assembled with said bone implant member between said washer and said body portion of said receiver member, and form a first open configuration, said first configuration including said first surface of said washer positioned adjacent said second surface of said head portion and said first surface of said head portion being positioned adjacent said body portion of said receiver member for contacting an elongated member in said passage of said receiver member, said first configuration including said extension portion being received in said passage of said head portion, extending beyond said second surface of said head portion and engaging said washer, said receiver member being rotatable relative to said bone implant member along said connection axis prior to locking the system;
wherein said receiver member, said bone implant member and said washer are lockable to form a second locked configuration, said second configuration including said closure member being received in said aperture to substantially lock an elongated member in said passage of said receiver member and substantially lock said receiver member at a desired angular position relative to said bone implant member.

14. The system of claim 13, wherein said second surface of said head portion includes radial splines and said first surface of said washer includes radial splines, wherein said radial splines of said head portion are configured to engage said radial splines of said washer to substantially prevent rotation of said washer relative to said head portion.

15. The system of claim 13, wherein said washer includes a hole extending from said first surface toward said second surface, said hole including threads and being configured to receive at least part of said extension portion, wherein said extension portion of said receiver member includes external threads configuring to engage said threads of said hole along said connection axis.

16. The system of claim 13, wherein said closure member is a threaded set screw and said aperture includes internal threads configured to engage said threaded set screw.

17. A spinal fixation system, comprising
a bone implant member having a bone engaging portion and a head portion; and
a receiver member engaged with a washer, such that said washer rotates with said receiver member relative to said bone implant member prior to locking the system, wherein said receiver member is positioned adjacent a first side of said head portion of said bone implant member and said washer is positioned adjacent a second side of said head portion of said bone implant member;
wherein said receiver member includes a body portion and a threaded extension portion, said extension portion extending along a connection axis, wherein said body portion defines a passage extending along a longitudinal axis to receive part of an elongated member, said connection axis being transverse to said longitudinal axis, wherein said body portion further defines at least one aperture in communication with said passage to receive a closure member to lock an elongated member in said passage;
wherein said head portion of said bone implant member defines a passage configured to receive part of said extension portion, said extension portion extending through said passage beyond said second side of said head portion to engage said washer, said second side of said head portion including radial splines; and
wherein said washer includes a first side and a second side, said first surface of said washer including radial splines configured to engage said radial splines of said head portion, wherein said washer defines a threaded hole extending therethrough along said connection axis, said threaded extension portion configured to be threadingly received in said threaded hole.

18. The system of claim 17, wherein said washer includes at least one indentation adjacent said threaded hole at said second side of said washer, and wherein said threaded extension portion is configured to be staked to said threaded hole at said indentation.

19. The system of claim 17, comprising a closure member configured to be received in said aperture to substantially lock an elongated member in said passage of said receiver member and substantially lock said receiver member at a desired angular position relative to said bone implant member.

20. The system of claim 17, wherein said aperture extends along a closure axis, and wherein said aperture is angled such that said closure axis is oblique to said connection axis.
